(19) United States
(12) Patent Application Publication
(10) Pub. No.: US 2001/0011543 A1
(43) Pub. Date: Aug. 9, 2001

(54) Controlled food flow in a patient

(76) Inventor: Peter Forsell, Menzingen (CH)

Correspondence Address: NIXON & VANDERHYE P.C., 8th Floor, 1100 North Glebe Road, Arlington, VA, 22201-4714, US

(21) Appl. No.: 09/753,656

(22) Filed: Jan. 4, 2001

Related U.S. Application Data
(63) Continuation-in-part of application No. 09/373,224, filed on Aug. 12, 1999, now abandoned.

(51) Int. Cl. A61B 19/00

(52) U.S. Cl. 128/899; 604/909; 606/191

(57) ABSTRACT

In the treatment of morbid obesity or heartburn and reflux disease an elongated restriction member (12) is formed in a substantially closed loop around a human's stomach or esophagus to form a stoma opening in the stomach or esophagus. The size of the stoma opening is adjustable by an implanted adjustment device. A control device (22) is utilized to control the adjustment device, in order to either reduce or enlarge the size of the stoma opening, for example in response to the time of the day. Nausea in a treated obese human can be minimized or substantially eliminated as a result of the control device controlling the adjustment device to keep the stoma opening substantially fully open between meals (such as at night when the human is sleeping).

A sensor (23), such as a pressure or position sensor, is surgically implanted in the human's body so that the sensor may either directly or indirectly sense a physical parameter of the human, such as the pressure in the stomach or the human's orientation with respect to the horizontal. If in response to sensing by the sensor it is determined by the control device that a significant change in the physical parameter has occurred, then the control device controls the adjustment device to either reduce or enlarge the size of the stoma opening.

This application is a continuation-in-part of U.S. Ser. No. 09/373,224, filed Aug. 12, 1999.

### BACKGROUND OF THE INVENTION

The present invention relates to an apparatus and procedure for controlling the food flow through the stomach or esophagus of a patient.

This kind of apparatus in the form of a gastric banding device, in which a band encircles a portion of a patient's stomach to restrict the food intake of the patient, have been used in surgery for morbid obesity to form a small gastric pouch above the band and a reduced stoma opening in the stomach. Although such a band is applied around the stomach to obtain an optimal stoma opening during surgery, some prior gastric banding devices are provided with an adjustment means enabling a minor post-operation adjustment of the size of the stoma opening. In all such prior art devices, such as disclosed in U.S. Pat. No. 4,592,339, European Patent No. 0611561 and International Patent Application WO 94/27504, the adjustment means typically comprises an inflatable cavity in the band and an injection port in fluid connection with the inflatable cavity. The injection port is subcutaneously implanted to allow the addition of fluid to or withdrawal of fluid from the cavity by an injection needle penetrating the patient's skin and passing into the injection port. In practice, the band is made of silicone which is a material approved for implantation and the fluid is a liquid such as an isotonic salt solution mixed with other conventional materials.

Thus, the only way for a patient carrying a gastric band of the type used in the above-discussed prior art devices to have the stoma opening adjusted is by visiting a doctor or nurse who is able to use an injection needle for withdrawing some liquid from or adding some liquid to the band. However, a problem with the prior art devices is that when the patient lies in bed sleeping it may happen that the stoma opening is completely closed by the stomach wall, which might cause the patient to vomit and feel sick. In such a situation the patient normally has no doctor or nurse available.

U.S. Pat. No. 5,938,669 discloses an adjustable hydraulic gastric banding device, which can be remote controlled by a doctor to adjust the stoma opening in the stomach. An implanted pressure sensor is provided to indicate the pressure in an implanted hydraulic tube of the gastric banding device. An alarm signal is produced if the pressure sensor senses a pressure that departs from a predetermined range. An implanted battery supplies energy to implanted energy consuming components of the gastric banding device.

The kind of apparatus discussed above for forming a stoma opening in the stomach or esophagus of a patient has also been used for treating heartburn and reflux disease due to hiatal hernia, i.e. a portion of the stomach immediately below the gastric fundus slides upwardly through the esophageal hiatus. In consequence, stomach acids and foods are regurgitated into the esophagus. In the late 1970s a prior art prosthesis called Angelchik, according to U.S. Pat. No. 3,875,928, was used to operatively treat heartburn and reflux disease. However, the Angelchik prosthesis had a major disadvantage in that it was not possible to adjust the size of the stoma opening after the operation. A further disadvantage was that the prosthesis did not satisfactorily protect the esophagus and the surrounding area against injuries due to poor shape of the prosthesis. Therefore, operations using the Angelchik prosthesis are no longer practised.

An operation technique, semi-fundoduplicatio, is currently in use for treating heartburn and reflux disease. A most common operation is Nissen semi-fundoduplicatio, in which one takes the fundus of the stomach and makes a three quarter of a turn around the esophagus and sutures between the stomach and esophagus. Although this operation works fairly well it has three main disadvantages. Firstly, most patients treated in accordance to semi-fundoduplicatio lose their ability to belch. Secondly, many of these patients get dysphagia, i.e. difficulties to swallow after the operation. Thirdly, it is not possible to adjust the stoma opening in the esophagus or stomach in any way after the operation. Characteristic for these patients is the variation of their problems over the day. For example, many patients have difficulties during the night when they lie down because of stomach acid leaking up into the esophagus.

### SUMMARY OF THE INVENTION

The prime object of the present invention is to provide a new general apparatus for controlling the food flow in the stomach or esophagus of a patient, wherein the new apparatus is suited for treating obese patients as well as patients suffering from heartburn and reflux disease.

Another object of the present invention is to provide a new convenient apparatus for controlling the food flow in the stomach or esophagus of a patient who suffers from obesity or hearburn and reflux disease, which practically eliminates the patient's need for visiting a doctor or nurse in order to adjust the stoma opening so that the patient always is satisfied.

According to one aspect of the present invention there is provided an apparatus for controlling the food flow in the stomach or esophagus of a patient, the apparatus comprising an implanted adjustable restriction device engaging the patient's stomach or esophagus to form a stoma opening in the stomach or esophagus, an implanted adjustment device for adjusting the restriction device in the loop to change the size of the stoma opening, an implanted sensor for sensing at least one physical parameter associated with the patient, and a control device which controls the adjustment device to adjust the restriction device to change the size of the stoma opening in response to the sensor sensing a change in the physical parameter.

As an alternative or in combination with the sensor, the control device may control the adjustment device in response to the time of the day. In this case the control device may comprise a clock mechanism used for controlling the adjustment device to adjust the restriction device to keep the stoma opening at different sizes during different time periods of the day.

The sensor may sense the pressure in the patient's stomach or esophagus, or the orientation of the patient with respect to the horizontal.

The control device may comprise an implanted internal control unit for directly controlling the adjustment device in response to signals from the sensor. Alternatively or in combination, the control device may comprise an external control unit outside the patient's body for directly or indirectly controlling the adjustment device in response to signals from the sensor. The external control unit may store information on the physical parameter sensed by the sensor and be manually operated to control the adjustment device based on the stored information.

Conveniently, the apparatus may further comprise at least one implanted sender for sending information on the physical parameter sensed by the sensor.

In accordance with a particular embodiment of the invention, the sensor comprises a pressure sensor for sensing as the physical parameter the pressure in the patient's stomach or esophagus. The pressure sensor may be any suitable known or conventional pressure sensor such as shown in U.S. Pat. Nos. 5,540,731, 4,846,181, 4,738,267, 4,571,749, 4,407,296 or 3,939,823; or an NPC-102 Medical Angioplasty Sensor.

Preferably, the control device controls the adjustment device to change the size of the stoma opening in response to the pressure sensor sensing a change in the pressure in the stomach or esophagus.

In the case of treating obese patients, in accordance with one embodiment of the invention the control device controls the adjustment device to reduce the stoma opening in response to the pressure sensor sensing a pressure, within a normal pressure range, equal to or exceeding a predetermined value, and to enlarge the stoma opening in response to the pressure sensor sensing a pressure below the predetermined value. The predetermined value is the pressure that normally occurs preferably in the stomach or, alternatively, in the esophagus soon after the patient has started to eat. Thus, food reaching the stomach causes an increase in the pressure in the stomach.

Between meals, for example at night, when the stomach is empty and the sensed pressure is well below the predetermined high pressure, the adjustment device can keep the stoma opening as large as possible, i.e. substantially fully open. As a result, the stoma opening will be relatively large, which minimizes the risk of the stoma opening closing completely. When the obese patient eats, so that the food entering the stomach increases the pressure therein, the stoma opening is reduced when the pressure sensor senses the predetermined value. Consequently, the obese patient's hunger is soon satisfied after a relatively small intake of food, which will lead to a reduction of the obese patient's weight.

In the case of treating patients suffering from heartburn and reflux disease, in accordance with another embodiment of the invention, the control device controls the adjustment device to enlarge the stoma opening in response to the pressure sensor sensing a pressure, within a normal pressure range, equal to or exceeding a predetermined value, and to reduce or close the stoma opening in response to the pressure sensor sensing a pressure below the predetermined value. The predetermined value is the pressure that occurs preferably in the esophagus or, alternatively, in the stomach soon after the patient has started to eat. Thus, food reaching the esophagus (or stomach) causes an increase in the pressure in the esophagus (or stomach).

Between meals, for example at night, when the stomach is empty and the sensed pressure is well below the predetermined value, the control device controls the adjustment device to adjust the restriction device to restrict or close the stoma opening. Consequently, the restriction device will work as an artificial sphincter.

In addition to or as an alternative to the two embodiments described above related to the treatment of obese patients and patients suffering from heartburn and reflux disease in which normal pressures in the stomach or esophagus are sensed, the control device may be adapted to control the adjustment device to enlarge the stoma opening in response to the pressure sensor sensing a pressure equal to or exceeding a too high value which is injurious to the patient. For example, an injurious pressure can occur in the stomach or esophagus if a large piece of food get stuck in the stoma opening. By enlarging the stoma opening the piece of food will be able to pass through.

Conveniently, the pressure sensor may indirectly sense the pressure in the stomach by sensing the pressure exerted by the stomach or esophagus against the restriction device.

In accordance with a particular embodiment of the invention, the sensor comprises a position sensor for sensing as the physical parameter the orientation of the patient with respect to the horizontal. The position sensor may be a biocompatible version of what is shown in U.S. Pat. Nos. 4,942,668 and 5,900,909.

In the case of treating an obese patient, the control device controls the adjustment device to increase the stoma opening in response to the position sensor sensing that the patient has assumed a substantially horizontal orientation, i.e. that the patient is lying.

In the case of treating a patient suffering from heartburn and reflux disease, the control device controls the adjustment device to reduce or close the stoma opening in response to the position sensor sensing that the patient has assumed a substantially horizontal orientation, i.e. that the patient is lying.

Alternatively, the apparatus may further comprise such a position sensor in addition to the above described pressure sensor.

As mentioned above, a clock mechanism may be used for controlling the adjustment device to adjust the restriction device to keep the stoma opening at different sizes during different time periods of the day. In case a sensor of any of the above described types (pressure or position sensor) is provided, the clock mechanism is used for controlling the adjustment device provided that the physical parameter sensed by the sensor does not override the clock mechanism. Preferably, the control device comprises an internal control unit implanted in the patient and a wireless remote control adapted to set control parameters of the internal control unit from outside the patient. At least one of the control parameters, which is settable by the wireless remote control, is associated with the physical parameter. Suitably, the wireless remote control may set the above mentioned clock mechanism.

The wireless remote control may be capable of transforming wireless energy from a signal transmitted by the remote control into energy for powering implanted energy consuming components of the apparatus. For example the wireless remote control may comprise a signal (e.g. electromagnetic or sound waves, magnetic energy, digital pulses, etc.) transmitter, an implanted signal receiver, and an implanted energizer unit for transforming wireless energy from the signal, as it is transmitted from the transmitter to the signal receiver, into said energy (typically different than the wireless energy) for powering implanted energy consuming components of the apparatus, such as the adjustment device and/or the sensor. The wireless signal may comprises a wave signal, for example an electromagnetic wave signal, such as an infrared light signal, a visible light signal, an ultra violet light signal, a laser signal, a micro wave signal, a radio wave signal, an x-ray radiation signal, and a gamma radiation signal. Where applicable, one or more of the above signals may be combined. Alternatively, the wave signal may comprise a sound wave signal, such as an ultrasonic signal. Generally, the wireless signal may comprise a digital, analog or a digital and analog signal.

Alternatively, the apparatus may comprise an implanted battery or accumulator, such as a capacitor, for energizing the adjustment device and/or the sensor.

The adjustment device may comprise an expandable cavity in the restriction device, the size of the stoma opening being reduced upon expansion of the cavity and increased upon contraction of the cavity, and a reservoir for hydraulic fluid (e.g. a salt solution). In this case the adjustment device is adapted to distribute hydraulic fluid from the reservoir to expand the cavity, and to distribute hydraulic fluid from the cavity to the reservoir to contract the cavity, to thereby change the size of the stoma opening. The reservoir may be attached or fixed to the restriction device, or integrated therewith. Furthermore, the adjustment device may comprise a pump for pumping fluid between the cavity and the reservoir. The pump is suitably subcutaneously implanted in the patient remote from the restriction device. Alternatively, the pump may be attached or fixed to the restriction device.

In accordance with a preferred embodiment of the invention, the reservoir, pump and restriction device form a single piece, suitably together with the sensor.

Alternatively, the restriction device may be non-inflatable, which has the advantage that the risk of fluid leaking from the restriction device is avoided. In this case it is preferred to use an adjustment device which is designed to mechanically adjust the non-inflatable restriction device.

Suitably, an implanted battery or accumulator, such as a capacitor, may be provided for energizing the adjustment device and/or the sensor.

The invention is not limited to sensing the pressure in the patient's stomach or esophagus, or the patient's orientation with respect to the horizontal, but may sense a wide variety of other physical parameters associated with the patient, such as parameters associated with rest or sleep, etc.

In accordance with another aspect of the present invention there is provided a method of controlling the food flow in the stomach or esophagus of a patient, comprising: (a) Surgically implanting in the patient an adjustable restriction device engaging the patient's stomach or esophagus to form a stoma opening in the stomach or esophagus. (b) Surgically implanting in the patient an adjustment device which adjusts the restriction device and a sensor for sensing at least one physical parameter associated with the patient. And (c) controlling the adjustment device to adjust the restriction device to change the size of the stoma opening in response to the sensor sensing a change in the physical parameter.

Where the method is practiced on a patient suffering from morbid obesity, the sensor may comprise a pressure sensor for directly or indirectly sensing as the physical parameter the pressure in the stomach or esophagus and (c) may be practiced to reduce the stoma opening when the pressure is at a pressure value commonly occurring when the patient eats and to enlarge the stoma opening when the pressure is at a pressure value commonly occurring between meals. Conveniently, (c) may be practiced to substantially fully open the stoma opening when the pressure is at a pressure value commonly occurring when the patient is sleeping at night. Also, (c) may be practiced to substantially fully open the stoma opening when the pressure sensor senses a too high pressure, to avoid that injurious pressures arise in the stomach or esophagus. The method may further comprise (d) controlling the adjustment device in response to the time of the day to vary the stoma opening. Method step (d) may be practiced unless overridden by the pressure sensor, for example when the pressure sensor senses pressure that would cause the stoma opening to be reduced or substantially closed.

Alternatively, in the method the adjustment device may be implanted in the patient's torso, and the sensor may comprise a position sensor for sensing as the physical parameter the orientation of the patient's torso with respect to the horizontal, wherein (c) is practiced to enlarge the stoma opening when the position sensor senses a substantially horizontal orientation of the patient's torso. The method may further comprise controlling the adjustment device in response to the time of the day to vary the stoma opening unless overridden by the position sensor.

Where the method is practiced on a patient suffering from heartburn and reflux disease, the sensor may comprise a pressure sensor for directly or indirectly sensing as the physical parameter the pressure in the stomach or esophagus, wherein (c) is practiced to enlarge the stoma opening when the pressure is at a pressure value commonly occurring when the patient eats and to reduce or close the stoma opening when the pressure is at a pressure value commonly occurring between meals or when the patient is sleeping at night. Also, (c) may be practiced to substantially fully open the stoma opening when the pressure sensor senses a too high pressure, to avoid that injurious pressures arise in the stomach or esophagus. The method may further comprise (d) controlling the adjustment device in response to the time of the day to vary the stoma opening. Method step (d) may be practiced unless overridden by the pressure sensor, for example when the pressure sensor senses a pressure that would cause the stoma opening to be enlarged.

Alternatively, in the method practiced on a patient suffering from heartburn and reflux disease, the adjustment device may be implanted in the patient's torso, and the sensor may comprise a position sensor for sensing as the physical parameter the orientation of the patient's torso with respect to the horizontal, wherein (c) is practiced to restrict or close the stoma opening when the position sensor senses a substantially horizontal orientation of the patient's torso. The method may further comprise (d) controlling the adjustment device in response to the time of the day to vary the stoma opening unless overridden by the position sensor.

By using the apparatus of the present invention for treating an obese patient it is possible to improve the quality of life of the patient. Thus, the present invention also provides a method of improving the quality of life of an obese patient having an adjustable restriction device engaging the patient's stomach or esophagus to form a stoma opening in the stomach or esophagus. The method comprises

(a) surgically implanting an adjustment device which adjusts the restriction device and a sensor in the patient operatively associated with the stoma opening;

(b) sensing at least one physical parameter of the patient using the sensor; and

(c) controlling the adjustment device to enlarge the stoma opening in response to the sensor sensing a significant change in the physical parameter.

In the method (b) may be practiced by sensing the pressure in the patient's stomach, and (c) may be practiced so that if the pressure in the patient's stomach is below a predetermined value then the adjustment device is controlled to enlarge the stoma opening. Furthermore, (b) may be practiced by sensing the pressure in the patient's stomach, and (c) may be practiced so that if the pressure in the patient's stomach is above a predetermined value then the adjustment device is controlled to reduce the stoma opening.

Alternatively, (b) may be practiced by sensing the orientation of the patient with respect to the vertical, and (c) may be practiced so that if the patient is substantially horizontal then the adjustment device is controlled to enlarge the stoma opening.

Also, (b) and (c) may be practiced to substantially fully open the stoma opening when the pressure in the stomach is at a pressure value commonly occurring when the patient is sleeping at night.

An internal control unit of the control device may be implanted in the patient at substantially the same time as the sensor, so that the internal control unit is mounted on the restriction device, or at some other location associated with the implant. Suitably, the internal control unit is operated exteriorly of the patient in a non-invasive manner to control the adjustment device.

In accordance with the invention, there is also provided a method of controlling the food flow through the stomach or esophagus of a patient comprising:

in a laparascopic surgery procedure insufflating the abdomen of the patient to form a pneumoperitoneum;

introducing at least one laparascopic trocar into the abdomen;

introducing an adjustable restriction device, an adjustment device for adjusting the restriction device and a sensor for sensing at least one physical parameter associated with the patient into the abdomen;

placing the adjustment device, sensor and adjustable restriction device in the patient's abdomen, so that the restriction device engages the patient's stomach or esophagus to form a stoma opening in the stomach or esophagus; and

controlling the adjustment device to adjust the restriction device to change the size of the stoma opening in response to the sensor sensing a change in the physical parameter.

### BRIEF DESCRIPTION OF THE DRAWINGS

FIG. 1 is a schematic perspective view of the torso of an obese human having an apparatus according to the invention, showing internal body portions of the human schematically for clarity of illustration;

FIG. 2 is a schematic side view, with portions cut away for clarity of illustration, of an exemplary apparatus according to the invention used in the human body as illustrated in FIG. 1; and

FIG. 3 is a view like that of FIG. 2 only of another exemplary embodiment according to the invention.

### DETAILED DESCRIPTION OF THE DRAWINGS

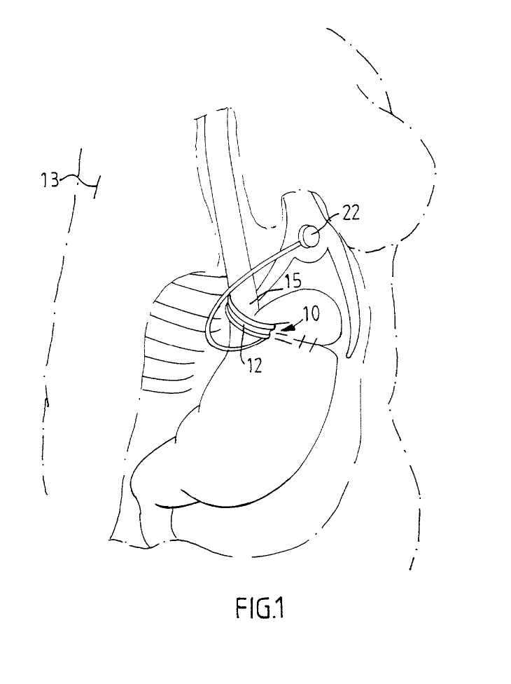

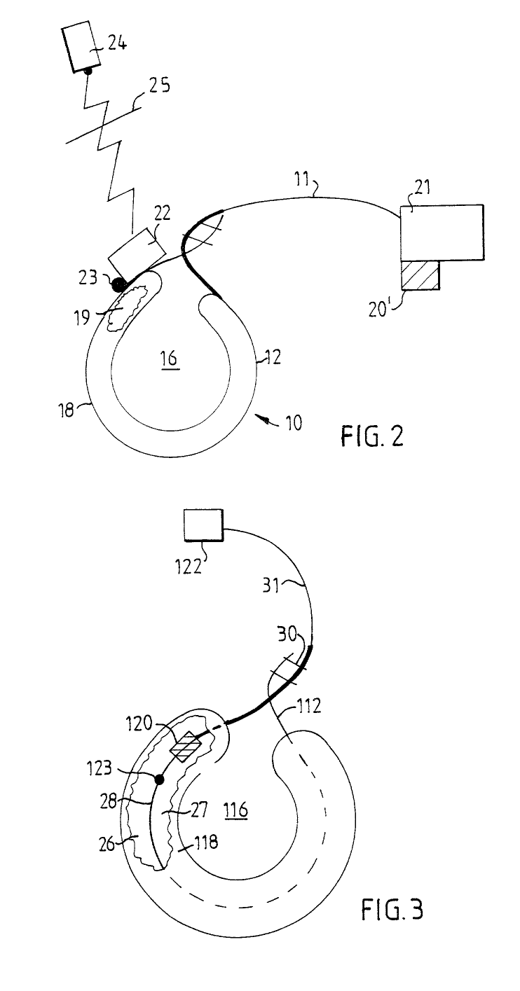

An apparatus according to the present invention for controlling the food flow in the stomach (or esophagus) of an obese human patient is illustrated schematically at 10 in FIGS. 1 and 2. The apparatus 10 includes an adjustable restriction device in the form of an elongated restriction member 12, such as a gastric band, which is surgically implanted in the human body 13 around the human's stomach 14 or—as shown in the embodiment of FIG. 1—both the stomach and esophagus 15 to form a stoma opening between an upper small pouch of the stomach 14 and a lower major portion of the stomach 14. The elongated restriction member 12 is formed into a substantially closed loop, the loop defining a restriction opening and a corresponding stoma opening in the stomach, such as illustrated schematically at 16 in FIG. 2 (FIG. 2 shows the restriction opening 16 dimensioned, compared to the illustration in FIG. 1, in a manner whereby it would be disposed around the stomach 14 rather than the esophagus 15).

In the case of treating a human patient suffering from heartburn and reflux disease, the restriction member 12 would be applied around the esophagus or around an upper portion of the stomach close to the cardia without forming the upper pouch of the stomach illustrated in FIG. 1.

The apparatus 10 includes an adjustment device, which may be of any suitable type. In the embodiment illustrated in FIG. 2 the adjustment device comprises an expandable element 18 integrated with the band 12 and defining an interior cavity 19. An implanted pump 20 remote from the band 12 is connected thereto via a fluid conduit 11. The pump 20 is fixed to a reservoir, likewise implanted, for hydraulic fluid, shown generally at 21 in FIG. 2. The hydraulic fluid would be any suitable substantially incompressible fluid, which will not cause severe illness or injury to the human if it were to leak from the reservoir 21 or the cavity 19, such as a salt solution. By pumping hydraulic fluid from the reservoir 21 through the conduit 11 into the cavity 19, the pump 20 causes the element 18 to expand thereby reducing the size of the opening 16, whereas by pumping hydraulic fluid out of the cavity 19 into the reservoir 21 the pump 20 causes the element 18 to contract, causing the opening 16 to enlarge.

The pump 20 may be controlled by a control unit 22. While the control unit 22 may be mounted exteriorly of the body 13, in the preferred embodiment the control unit 22 is mounted within the body 13, preferably on the elongated restriction member 12, and adjacent the cavity 19. Electrical interconnections (not shown) are provided between the control unit 22 and the pump 20. A battery for operating the control unit 22 and pump 20 may be provided right within the control unit 22. Alternatively, a power source for powering the control unit 22 and the pump 20 may be located exteriorly of the body 13. Energy from such an exterior power source may be wirelessly transmitted to implanted energy consuming components.

A pressure sensor 23, shown schematically in FIG. 2, is implanted in the body 13 of the human patient for sensing the pressure in the stomach 14. For example, in the embodiment illustrated in FIG. 2 the pressure sensor 23 is mounted on the restriction member 12 and indirectly senses the pressure in the stomach 14 by sensing the pressure exerted by the stomach against the expandable element 18. However, the sensor 23 may be mounted directly on the inner side of the elongated restriction member 12 at a location remote from the cavity 19 to directly abut the stomach, or any other suitable mounting may be provided as long as the pressure sensor 23 is able to sense the pressure or related value within stomach 14 that is caused when food is ingested by the human patient.

The sensor 23 may be any suitable known or conventional sensor which is capable of performing the functions as set forth above. Some non-limiting examples of implantable sensors include those described in U.S. Pat. Nos. 5,540,731, 4,846,181, 4,738,267, 4,571,749, 4,407,296 or 3,939,823, and the NPC-102 Medical Angioplasty Sensor.

Alternatively the adjustment device may mechanically adjust the restriction member 12, e.g. a motor may be provided to adjust member 12.

The control unit 22 may be of the type which communicates effectively with a wireless remote control 24 illustrated schematically in FIG. 2, with the zig-zag line between the elements 22, 24 indicating wireless communication therebetween, and the solid cross line 25 indicating that the remote control 24 is exterior of the body 13. The remote control 24 may be for setting control parameters of the control unit 22 from outside the body 13 without mechanically penetrating the human patient. One of the control parameters which is settable by the device 24 may be the predetermined pressure values that the sensor 23 senses and communicates to the control unit 22 (either by electrical connections, or in a wireless manner) to cause the pump 20 to operate and hydraulic fluid to be removed from or pumped into the cavity 19. Wireless energy carrying signals from the remote control 24 may be electromagnetic or sound or other types of waves, magnetic transfer, or digital pulses.

In one embodiment of the invention the control unit 22 may include a clock mechanism mounted on the restriction member 12 and used for controlling the adjustment device 17 to adjust the restriction member 12 to keep the stoma opening 16 at different sizes during different time periods of the day, provided that the pressure sensed by the pressure sensor 23 does not exceed a predetermined value (which would indicate food in the stomach 14). For example in the middle of the night when it is expected that the obese human would have little or no food in his/her stomach 14, the control unit 22 would automatically operate the pump 20 to pump fluid out of the cavity 19 into the reservoir 21 so as to substantially fully open the stoma opening 16, whereas at other times of the day the pump 20 could be controlled to vary the size of the stoma opening 16 from the maximum to a minimum value.

The remote control 24 may be used to set the clock mechanism 22.

For example, the wireless remote control 24 may comprise a signal transmitter, and a signal receiver may be implanted within the body 13 (e.g. as part of the control unit 22), and an energizer unit may also be implanted in the body 13 (e.g. as part of the control unit 22) for transforming wireless energy from the signals as they are transmitted to the signal receiver into energy different than the wave energy, for example electrical energy, for energizing the adjustment device (e.g. by operating the pump 20) and sensor 23. Alternatively a battery may be implanted in the body 13 (e.g. as part of the control unit 22) for energizing the adjustment device (e.g. the pump 20) and sensor 23, or an accumulator (such as a capacitor) may be implanted in the patient for energizing the adjustment device and sensor 23.

The present invention also provides a method for minimizing or eliminating nausea in an obese human having an apparatus 10, as a result of the stoma opening 16 substantially closing between meals. The method comprises: (a) Implanting (e.g. with a conventional surgical procedure) the adjustment device and pressure sensor 23 in the obese human's body 13 operatively associated with the stoma opening 16. (b) Sensing the pressure in the stomach 14; and (c) if in response to (b) it is determined that the pressure is below a predetermined value (indicating little or no food in the stomach 14), then controlling the adjustment device to substantially fully open the stoma opening 16, so that nausea is minimized or substantially eliminated. In the method (b) and (c) may be practised to substantially fully open the stoma opening 16 when the pressure in the stomach is at a pressure value commonly occurring when the human is sleeping at night.

The method may further comprise implanting the control unit 22 in the human's body 13 at substantially the same time that (a) is practised so that the control unit 22 is mounted on the restriction member 12 or at some other location associated with the implant, and operating the control unit 22 exteriorly of the human in a non-invasive manner (as by using the remote control 24) to control the adjustment device.

Furthermore, the invention provides a method of treating morbid obesity in a human comprising: (a) Surgically implanting (preferably in a laparascopic surgery) in the human an elongated restriction member 12 defining a substantially closed loop (see FIG. 2) around the human's stomach 14 or esophagus 15, defining a stoma opening 16. (b) Surgically implanting in the human an adjustment device which adjusts the stoma opening 16, and a pressure sensor 23 for sensing the pressure in the humans' stomach 14. (c) In response to sensing by the pressure sensor 23 of a pressure in the human's stomach 14 greater than a predetermined amount, controlling the adjustment device to reduce the size of the opening 16. And (d) in response to sensing by the pressure sensor 23 of a pressure less than a predetermined amount in the human stomach 14, controlling the adjustment device to substantially fully open the opening 16. The method may also comprise (e) controlling the adjustment device in response to the time of day (e.g. using a clock mechanism as described above, e.g. as part of the control unit 22) to vary the stoma opening 16 unless overridden by the pressure sensor 23 sensing pressure in the stomach 14 that would cause the stoma opening to be less than fully open, or “closed” (that is having a minimize size substantially preventing further passage of food particles into the stomach, or a part thereof).

FIG. 3 shows an embodiment similar to that of FIG. 2 with comparable components shown by the same reference numbers only preceded by a “1”. In the FIG. 3 embodiment, however, two chambers 26,27 are separated from each other in a fluid tight manner by a partition wall 28, and the pump 120 pumps fluid from one chamber 26 to the other chamber 27 to change the size of the stoma opening 116. The sensor 123 may in this case be a conventional position sensor, which senses the orientation of the patient with respect to the horizontal. Both the pump 120 and sensor 123 are fixed to the partition wall 28 inside the chambers 26,27. Conventional locking members 30 may be used to hold the elongated restriction member 112 in the formed loop. The control unit 122 is implanted remote from the restriction member 112 and operably connected to the sensor 123 and pump 120 through a line 31. As in the other embodiments the control unit 122 may include an internal clock mechanism, and may be controlled from externally of the human by wireless remote control (like the remote control 24 in FIG. 2). If desired a pressure sensor 23 may also be included.

Alternative or additional sensors which sense other physical parameters related to sleep or rest of the human in which the sensors are implanted may also or alternatively be utilized.

While the invention has been herein shown and described in a preferred practical embodiment for the treatment of obese humans, it is to be understood by those of ordinary skill in the art that also patients suffering from heartburn and reflux disease can be treated with the apparatus 10 described, and many modifications may be made thereof within the scope of the invention, which scope is to be accorded the broadest interpretation of the appended claims so as to encompass all equivalent devices and methods.

What is claimed is:

1. An apparatus for controlling the food flow through the stomach or esophagus of a patient, the apparatus comprising:
an implanted adjustable restriction device engaging the patient's stomach or esophagus to form a restricted stoma opening in the stomach or esophagus,
an implanted adjustment device for adjusting said restriction device to change the size of the stoma opening,
an implanted sensor for sensing at least one physical parameter associated with the patient, and
a control device which controls said adjustment device to adjust said restriction device to change the size of the stoma opening in response to said sensor sensing a change in said physical parameter.

2. An apparatus according to claim 1, wherein said control device comprises an implanted internal control unit for directly controlling said adjustment device in response to signals from said sensor.

3. An apparatus according to claim 1, wherein said control device comprises an external control unit outside the patient's body for controlling said adjustment device in response to signals from said sensor.

4. An apparatus according to claim 3, wherein said external control unit directly controls said adjustment device in response to signals from said sensor.

5. An apparatus according to claim 3, wherein said external control unit stores information on said physical parameter sensed by said sensor and is manually operated to control said adjustment device based on said stored information.

6. An apparatus according to claim 1, wherein said control device comprises an implanted internal control unit and an external control unit outside the patient's body, for controlling said adjustment device in response to signals from said sensor.

7. An apparatus according to claim 1, further comprising at least one implanted sender for sending information on said physical parameter sensed by said sensor.

8. An apparatus according to claim 1, wherein said sensor comprises a pressure sensor for sensing as said physical parameter the pressure in the patient's stomach or esophagus.

9. An apparatus according to claim 8, wherein said pressure sensor indirectly senses the pressure in the stomach or esophagus by sensing the pressure exerted by the stomach or esophagus against said restriction member.

10. An apparatus according to claim 8, wherein said control device controls said adjustment device to change the size of said stoma opening in response to said pressure sensor sensing a change in the pressure in the stomach or esophagus.

11. An apparatus according to claim 10, wherein the apparatus is intended for restricting the food intake of an obese patient, and said control device controls said adjustment device to reduce said stoma opening in response to said pressure sensor sensing a pressure equal to or exceeding a predetermined value.

12. An apparatus according to claim 10, wherein the apparatus is intended for restricting the food intake of an obese patient, and said control device controls said adjustment device to enlarge said stoma opening in response to said pressure sensor sensing a pressure below a predetermined value.

13. An apparatus according to claim 10, wherein the apparatus is intended for restricting the food intake of an obese patient, and said control device controls said adjustment device to enlarge said stoma opening in response to said pressure sensor sensing a pressure equal to or exceeding a too high value.

14. An apparatus according to claim 10, wherein the apparatus is intended for treating heartburn and reflux disease, and said control device controls said adjustment device to enlarge said stoma opening in response to said pressure sensor sensing a pressure equal to or exceeding a predetermined value.

15. An apparatus according to claim 10, wherein the apparatus is intended for treating heartburn and reflux disease, and said control device controls said adjustment device to reduce or close said stoma opening in response to said pressure sensor sensing a pressure below a predetermined value.

16. An apparatus according to claim 10, wherein the apparatus is intended for treating heartburn and reflux disease, and said control device controls said adjustment device to enlarge or fully open said stoma opening in response to said pressure sensor sensing a pressure equal to or exceeding a too high value, to avoid injurious high pressures in the stomach or esophagus.

17. An apparatus according to claim 1, wherein said sensor comprises a position sensor for sensing as said physical parameter the orientation of the patient with respect to the horizontal.

18. An apparatus according to claim 17, wherein the apparatus is intended for restricting the food intake of an obese patient, and said control device controls said adjustment device to increase said stoma opening in response to said position sensor sensing that the patient has assumed a substantially horizontal orientation.

19. An apparatus according to claim 17, wherein the apparatus is intended for treating heartburn and reflux disease, and said control device controls said adjustment device to restrict or close said stoma opening in response to said position sensor sensing that the patient has assumed a substantially horizontal orientation.

20. An apparatus according to claim 8, further comprising a position sensor for sensing the orientation of the patient with respect to the horizontal.

21. An apparatus according to claim 20, wherein the apparatus is intended for restricting the food intake of an obese patient, and said control device controls said adjustment device to increase said stoma opening in response to said position sensor sensing that the patient has assumed a substantially horizontal orientation.

22. An apparatus according to claim 20, wherein the apparatus is intended for treating heartburn and reflux disease, and said control device controls said adjustment device to restrict or close said stoma opening in response to said position sensor sensing that the patient has assumed a substantially horizontal orientation.

23. An apparatus according to claim 1, wherein said control device comprises a clock mechanism used for controlling said adjustment device to adjust said restriction member to keep said stoma opening at different sizes during different time periods of the day.

24. An apparatus according to claim 23, wherein said clock mechanism is used for controlling said adjustment device provided that said physical parameter sensed by said sensor does not override said clock mechanism.

25. An apparatus according to claim 1, wherein said control device comprises an implanted internal control unit and a wireless remote control which sets control parameters of said internal control unit from outside the patient.

26. An apparatus according to claim 25, wherein at least one of said control parameters, which is settable by said wireless control, is associated with said physical parameter.

27. An apparatus according to claim 25, wherein said internal control unit includes a clock mechanism used for controlling said adjustment device to adjust said restriction device to keep said stoma opening at different sizes during different time periods of the day.

28. An apparatus according to claim 27, wherein said wireless remote control is capable of setting said clock mechanism.

29. An apparatus according to claim 1, wherein said control device comprises a wireless remote control.

30. An apparatus according to claim 29, wherein said wireless remote control is transmits a signal and is capable of transforming wireless energy from said signal into energy for powering implanted energy consuming components of the apparatus.

31. An apparatus according to claim 30, wherein said wireless remote control comprises a signal transmitter for transmitting said signal, an implanted signal receiver, and an implanted energizer unit for transforming wireless energy from said signal, as they are transmitted from said signal transmitter to said signal receiver, into said energy for powering implanted energy consuming components of the apparatus.

32. An apparatus according to claim 30, wherein the energy transformed from said wireless energy is used for energizing said implanted sensor.

33. An apparatus according to claim 1, wherein said adjustment device comprises an expandable cavity in said restriction device and the size of said stoma opening is reduced upon expansion of said cavity and increased upon contraction of said cavity, and further comprising a reservoir for hydraulic fluid, said adjustment device distributing hydraulic fluid from said reservoir to expand said cavity and hydraulic fluid from said cavity to said reservoir to contract said cavity, to thereby change the size of said stoma opening.

34. An apparatus according to claim 33, wherein said reservoir is attached to said restriction device.

35. An apparatus according to claim 33, wherein said reservoir is fixed to said restriction device.

36. An apparatus according to claim 33, wherein said reservoir is integrated with said restriction device.

37. An apparatus according to claim 33, wherein said adjustment device comprises a pump for pumping fluid between said cavity and said reservoir.

38. An apparatus according to claim 37, wherein said pump is attached to said restriction device.

39. An apparatus according to claim 37, wherein said pump is fixed to said restriction device.

40. An apparatus according to claim 37, wherein said reservoir, pump and restriction device form a single piece.

41. An apparatus according to claim 37, wherein said reservoir, pump, sensor and restriction device form a single piece.

42. An apparatus according to claim 37, wherein said pump is subcutaneously implanted in the patient remote from said restriction device.

43. An apparatus according to claim 1, wherein said adjustment device mechanically adjusts said restriction device.

44. An apparatus according to claim 1, further comprising an implanted battery for energizing said adjustment device.

45. An apparatus according to claim 1, further comprising an implanted accumulator for energizing said adjustment device.

46. An apparatus according to claim 45, wherein said accumulator comprises a capacitor.

47. An apparatus according to claim 1, further comprising an implanted battery for energizing said sensor.

48. An apparatus according to claims 1, further comprising an implanted accumulator for energizing said sensor.

49. An apparatus according to claim 48, wherein said accumulator comprises a capacitor.

50. An apparatus for controlling the food flow through the stomach or esophagus of a patient, the apparatus comprising:
an implanted adjustable restriction device engaging the patient's stomach or esophagus to form a restricted stoma opening in the stomach or esophagus,
an implanted adjustment device for adjusting said restriction device to change the size of said stoma opening, and
a control device which controls said adjustment device to adjust said restriction device to change the size of said stoma opening in response to the time of the day.

51. An apparatus according to claim 50, wherein said control device comprises a clock mechanism used for controlling said adjustment device to adjust said restriction device to keep said stoma opening at different sizes during different time periods of the day.

52. An apparatus according to claim 50, wherein said control device comprises an implanted internal control unit and a wireless remote control which sets control parameters of said internal control unit from outside the patient.

53. An apparatus according to claim 52, wherein said internal control unit includes a clock mechanism used for controlling said adjustment device to adjust said restriction device to keep said stoma opening at different sizes during different time periods of the day.

54. An apparatus according to claim 53, wherein said wireless remote control is capable of setting said clock mechanism.

55. An apparatus according to claim 50, wherein said control device comprises a wireless remote control.

56. An apparatus according to claim 55, wherein said wireless remote control is transmits signals and is capable of transforming wireless energy from said signals into energy for powering implanted energy consuming components of the apparatus.

57. An apparatus according to claim 56, wherein said wireless remote control comprises a signal transmitter for transmitting said signals, an implanted signal receiver, and an implanted energizer unit for transforming wireless energy from said signals, as they are transmitted from said signal transmitter to said signal receiver, into said energy for powering implanted energy consuming components of the apparatus.

58. An apparatus according to claim 50, wherein said adjustment device comprises an expandable cavity in said restriction device and the size of said stoma opening is reduced upon expansion of said cavity and increased upon contraction of said cavity, and further comprising a reservoir for hydraulic fluid, said adjustment device distributing hydraulic fluid from said reservoir to expand said cavity and hydraulic fluid from said cavity to said reservoir to contract said cavity, to thereby change the size of said stoma opening.

59. An apparatus according to claim 58, wherein said reservoir is attached to said restriction device.

60. An apparatus according to claim 58, wherein said reservoir is fixed to said restriction device.

61. An apparatus according to claim 58, wherein said reservoir is integrated with said restriction device.

62. An apparatus according to claim 58, wherein said adjustment device comprises a pump for pumping fluid between said cavity and said reservoir.

63. An apparatus according to claim 62, wherein said pump is attached to said restriction device.

64. An apparatus according to claim 62, wherein said pump is fixed to said restriction device.

65. An apparatus according to claim 62, wherein said reservoir, pump and restriction device form a single piece.

66. An apparatus according to claim 62, wherein said pump is subcutaneously implanted in the patient remote from said restriction device.

67. An apparatus according to claim 50, wherein said adjustment device mechanically adjusts said restriction device.

68. An apparatus according to claim 50, further comprising an implanted battery for energizing said adjustment device.

69. An apparatus according to claim 50, further comprising an implanted accumulator for energizing said adjustment device.

70. An apparatus according to claim 69, wherein said accumulator comprises a capacitor.

71. A method of controlling the food flow through the stomach or esophagus of a patient comprising:
(a) surgically implanting in the patient an adjustable restriction device engaging the patient's stomach or esophagus to form a stoma opening in the stomach or esophagus;
(b) surgically implanting in the patient an adjustment device which adjusts the restriction device and a sensor for sensing at least one physical parameter associated with the patient; and
(c) controlling the adjustment device to adjust the restriction device to change the size of the stoma opening in response to the sensor sensing a change in the physical parameter.

72. A method as recited in claim 71, wherein (a) and (b) is practiced on a patient suffering from morbid obesity, and the sensor comprises a pressure sensor for directly or indirectly sensing as the physical parameter the pressure in the stomach or esophagus,

73. A method as recited in claim 72, wherein (c) is practiced to reduce the stoma opening when the pressure is at a pressure value commonly occurring when the patient eats and to enlarge the stoma opening when the pressure is at a pressure value commonly occurring between meals.

74. A method as recited in claim 72, wherein (c) is practiced to substantially fully open the stoma opening when the pressure is at a pressure value commonly occurring when the patient is sleeping at night.

75. A method as recited in claim 72, further comprising (d) controlling the adjustment device in response to the time of the day to vary the stoma opening.

76. A method as recited in claim 75, wherein (d) is practiced unless overridden by the pressure sensor

77. A method as recited in claim 72, wherein (c) is practiced to substantially fully open the stoma opening when the pressure sensor senses an exceedingly high pressure, to avoid that injurious pressures arise in the stomach or esophagus.

78. A method as recited in claim 71, wherein (a) and (b) is practiced on a patient suffering from morbid obesity and the adjustment device is implanted in the patient's torso, and the sensor comprises a position sensor for sensing as the physical parameter the orientation of the patient's torso with respect to the horizontal.

79. A method as recited in claim 78, wherein (c) is practiced to enlarge the stoma opening when the position sensor senses a substantially horizontal orientation of the patient's torso.

80. A method as recited in claim 78, further comprising (d) controlling the adjustment device in response to the time of the day to vary the stoma opening.

81. A method as recited in claim 80, wherein (d) is practiced unless overridden by the position sensor.

82. A method as recited in claim 71, wherein (a) and (b) is practiced on a patient suffering from heartburn and reflux disease, and the sensor comprises a pressure sensor for directly or indirectly sensing as the physical parameter the pressure in the stomach or esophagus,

83. A method as recited in claim 82, wherein (c) is practiced to enlarge the stoma opening when the pressure is at a pressure value commonly occurring when the patient eats and to reduce or close the stoma opening when the pressure is at a pressure value commonly occurring between meals.

84. A method as recited in claim 82, wherein (c) is practiced to restrict or close the stoma opening when the pressure is at a pressure value commonly occurring when the patient is sleeping at night.

85. A method as recited in claim 82, further comprising (d) controlling the adjustment device in response to the time of the day to vary the stoma opening.

86. A method as recited in claim 85, wherein (d) is practiced unless overridden by the pressure sensor.

87. A method as recited in claim 82, wherein (c) is practiced to substantially fully open the stoma opening when the pressure sensor senses an exceedingly high pressure, to avoid that injurious pressures arise in the stomach or esophagus.

88. A method as recited in claim 71, wherein (a) and (b) is practiced on a patient suffering from heartburn and reflux disease and the adjustment device is implanted in the patient's torso, and the sensor comprises a position sensor for sensing as the physical parameter the orientation of the patient's torso with respect to the horizontal.

89. A method as recited in claim 88, wherein (c) is practiced to restrict or close the stoma opening when the position sensor senses a substantially horizontal orientation of the patient's torso.

90. A method as recited in claim 89, further comprising (d) controlling the adjustment device in response to the time of the day to vary the stoma opening unless overridden by the position sensor.

91. A method of controlling the food flow through the stomach or esophagus of a patient comprising:
(a) surgically implanting in the patient an adjustable restriction device engaging the patient's stomach or esophagus to form a stoma opening in the stomach or esophagus;
(b) surgically implanting in the patient an adjustment device which adjusts the restriction device; and
(c) controlling the adjustment device to adjust the restriction device to change the size of the stoma opening in response to the time of the day.

92. A method as recited in claim 91, wherein (c) is practiced to keep the stoma opening at different sizes during different time periods of the day.

93. A method of improving the quality of life of an obese patient having an adjustable restriction device engaging the patient's stomach or esophagus to form a stoma opening in the stomach or esophagus, the method comprising:
(a) surgically implanting an adjustment device which adjusts the restriction device and a sensor in the patient operatively associated with the stoma opening;
(b) sensing at least one physical parameter of the patient using the sensor; and
(c) controlling the adjustment device to enlarge the stoma opening in response to the sensor sensing a significant change in the physical parameter.

94. A method as recited in claim 93, wherein (b) is practiced by sensing the pressure in the patient's stomach, and (c) is practiced so that if the pressure in the patient's stomach is below a predetermined value then the adjustment device is controlled to enlarge the stoma opening.

95. A method as recited in claim 93, wherein (b) is practiced by sensing the pressure in the patient's stomach, and (c) is practiced so that if the pressure in the patient's stomach is above a predetermined value then the adjustment device is controlled to reduce the stoma opening.

96. A method as recited in claim 93, wherein (b) is practiced by sensing the orientation of the patient with respect to the vertical, and (c) is practiced so that if the patient is substantially horizontal then the adjustment device is controlled to enlarge the stoma opening.

97. A method as recited in claim 94, wherein (b) and (c) are practiced to substantially fully open the stoma opening when the pressure in the stomach is at a pressure value commonly occurring when the patient is sleeping at night.

98. A method as recited in claim 93, further comprising implanting a control unit in the patient, and operating the control unit exteriorly of the patient in a noninvasive manner to control the adjustment device.

99. A method of controlling the food flow through the stomach or esophagus of a patient comprising:
in a laparascopic surgery procedure insufflating the abdomen of the patient to form a pneumoperitoneum;
introducing at least one laparascopic trocar into the abdomen;
introducing an adjustable restriction device, an adjustment device for adjusting the restriction device and a sensor for sensing at least one physical parameter associated with the patient into the abdomen;
placing the adjustment device, sensor and adjustable restriction device in the patient's abdomen, so that the restriction device engages the patient's stomach or esophagus to form a stoma opening in the stomach or esophagus; and
controlling the adjustment device to adjust the restriction device to change the size of the stoma opening in response to the sensor sensing a change in the physical parameter.
